(19) United States
(12) Patent Application Publication
(10) Pub. No.: US 2009/0324691 A1
(43) Pub. Date: Dec. 31, 2009

(54) METHODS AND OPHTHALMIC DEVICES USED IN THE TREATMENT OF OCULAR ALLERGIES

(76) Inventors: Shivkumar Mahadevan, Orange Park, FL (US); Edgar V. Menezes, Jacksonville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/482,676

(22) Filed: Jun. 11, 2009

Related U.S. Application Data
(60) Provisional application No. 61/076,847, filed on Jun. 30, 2008.

(51) Int. Cl. A61K 31/4535; A61P 27/14

(52) U.S. Cl. 424/429; 514/324

(57) ABSTRACT

Ophthalmic devices containing anti-allergic agents and methods of preparing the same are disclosed herein.

### RELATED APPLICATION

This application is a non-provisional filing of a provisional application, U.S. Ser. No. 61/076,847, filed on Jun. 30, 2008.

### FIELD OF THE INVENTION

This invention related to methods of adding anti-allergic agents to ophthalmic devices.

### BACKGROUND

Allergic conjunctivitis is a disease of the eye that affects millions of people. The symptoms of this disease include itchiness, tearing, and swelling of the eyes. Sometimes this disease is seasonally associated with the spring and summer hay fever seasons, but many people experience symptoms of this disease throughout the year. The symptoms of allergic conjunctivitis are caused and mediated by the binding of histamine to its receptor. Antihistamines are a class of pharmaceutical agent known to either or both suppress the release of histamine from associated mast cells and prevent the binding of histamine to its associated receptors. These agents have been used to treat the symptoms of allergic conjunctivitis and one such agent is ketotifen fumarate. Topical solutions of ketotifen fumarate are currently sold in the United States. The concentration ketotifen in of the U.S. approved ketotifen fumarate formulation is 0.025% (0.25 mg/mL). At that concentration, the recommended dosing regimen is twice daily. It is known that the recommended dosing can be reduced if the amount of ketotifen fumarate is increased, but it is also known that higher concentrations of ketotifen fumarate sting and burn upon initial administration to the eye.

Further ophthalmic lenses that contain antihistamines have been prepared. See U.S. patent application Ser. No. 11/686,979. These lenses deliver a minimum effective amount of antihistamines to the eye of a user over time. However, when the drug is delivered the ophthalmic lens is still usable by a wearer, even though it no longer contains enough antihistamine to treat the symptoms of allergic conjunctivitis. Since the ophthalmic lens may still be worn, it would be beneficial if a user could add additional antihistamine to a lens that has delivered that antihistamine to the eye of a user. Such a method and solutions that may be used are disclosed by the following invention.

### BRIEF DESCRIPTION OF THE DRAWING

FIG. 1 illustrates the uptake of ketotifen fumarate to an ophthalmic device over time.

### DETAILED DESCRIPTION OF THE INVENTION

This invention includes a method of preparing an ophthalmic device comprising an minimum effective amount of an anti-allergic agent comprising treating an ophthalmic device comprising less than about a minimum effective amount of an anti-allergic agent with a solution comprising said anti-allergic agent, wherein the amount of said anti-allergic agent in said solution exceeds the minimum effective amount. As used herein “anti-allergic agent” refers to chemical substances that alleviate the symptoms of allergic conjunctivitis. While not wishing to be bound by any particular mechanism of action, anti-allergic agents include but are not limited to chemical substances that inhibit the release of histamine, that block the binding of histamine to its receptors, inhibit mast cell production. Additional anti-allergic agents include but are not limited to decongestants, non-steroidal anti-inflammatory compound, and steroidal compounds. Particularly, examples of anti-allergic agents include but are not limited to acetmetacin, acrivastine, aldosterone, antazoline, astemizole, azatadine, azelastine, beclometasone, betamethasone, bromfenac, buclizine, carprofen, cetirizine, chloropyriline, chloropheniramine, clemastine, cromolyn, cyclizine, cyproheptadine, dexamethasone, diazoline, diclofenac, diphenhydramine, ebastine, emedastine, epinastine, etodolac, fenbufen, fenoprofen, fexofenadine, fludrocortisone, flurbiprofen, flurometalone, hydroxyzine, ibuprofen, indometacin, ketoprofen, ketorolac tromethamine, ketotifen, levocabastine, levoceterizine, lodoxamide, loratadine, loteprednol, loxoprofen, medrysone, mepivacaine, mequitazine, methdilazine, methapyrilene, nabumetone, naphazoline, naproxen, nedocromil, norastemizole, norebastine, olopatadine, phenidamine, phenylephrine, oxatamide, oxymetazoline, pemirolast, pheniramine, picumast, prednisilone, promethazine, rimexalone, repirinast, sulindac, suprofen, tetrahydozoline, terfenadine, tiaprofenic acid, tometim, tranilast, triamcinolone, trimeprazine, triprolidine, and pharmaceutically acceptable salts and mixtures thereof. Preferred anti-allergic agent include acrivatine, antazoline, astemizole, azatadine, azelastine, clemastine, cyproheptadine, ebastine, emedastine, fexofenadine, hydroxyzine, ketotifen, levocabastine, levoceterizine, mequitazine, methdialazine, methapyrilene, norastemizole, norebastine, picumast, promethazine, terfenadine, trimeprazine, triprolidine, and pharmaceutically acceptable salts and mixtures thereof. The class of substances known as antihistamines are the particularly preferred anti-allergic agents The particularly preferred antihistamines include, azelastine, epinastine, ketotifen, ketotifen fumarate, nor-ketotifen fumarate, olopatadine and mixtures thereof. More particularly preferred antihistamines include ketotifen, its pharmaceutically acceptable salts and mixtures thereof.

The term “minimum effective amount” refers to the weight of anti-allergic agent contained in an ophthalmic device prior to its use by a patient wherein such minimum effective amount alleviates the symptoms of allergic conjunctivitis. The minimum effective amount may vary depending upon the efficacy of a particular anti-allergic agent. For example, if the anti-allergic agent is ketotifen, the minimum effective amount is between greater than about 9 μg and about less than 90 μg, more particularly between about 40 μg and greater than about 9 μg, most preferably about 20 μg. It is preferred that minimum effective amount of anti-allergic agent other than ketotifen is an amount that exhibits an efficacy equivalent to or more efficacious greater than about 9 μg and about less than 90 μg, more particularly between about 40 μg and about 9 μg of ketotifen.

It is preferred that the minimum effective amount is exceeded by between about 0.1% and about 50%, in a volume of solution that is between about 500 μL and about 5000 μL preferably between about 10% and about 30%, in a volume of solution that is between about 250 μL and about 10,000 μL per lens, most preferably about 50% in a volume of solution that is about 1000 μL, per lens.

As used herein “treating” ( as well as treat) means physical methods of contacting the solution containing an anti-allergic agent and the ophthalmic device. Preferably treating refers to physical methods of contacting the anti-allergic agent with the ophthalmic devices at ambient temperature when the ophthalmic device is not it the eye of a patient. It is preferred that the ophthalmic device comprising less than the minimum effective amount is treated with the solution for greater than about 15 minutes to about 24 hours, more preferably greater than about two hours to about 16 hours, most preferably greater than about two hours to about 12 hours.

The “solutions” that are used in methods of this invention may be water-based solutions. Typical solutions include, without limitation, saline solutions, other buffered solutions, and deionized water. The preferred aqueous solution is deioinized water or saline solution containing salts including, without limitation, sodium chloride, sodium borate, sodium phosphate, sodium hydrogenphosphate, sodium dihydrogenphosphate, or the corresponding potassium salts of the same. These ingredients are generally combined to form buffered solutions that include an acid and its conjugate base, so that addition of acids and bases cause only a relatively small change in pH. The buffered solutions may additionally include 2-(N-morpholino)ethanesulfonic acid (MES), sodium hydroxide, 2,2-bis(hydroxymethyl)-2,2′,2″-nitrilotriethanol, n-tris(hydroxymethyl)methyl-2-aminoethanesulfonic acid, citric acid, sodium citrate, sodium carbonate, sodium bicarbonate, acetic acid, sodium acetate, ethylenediamine tetraacetic acid and the like and combinations thereof. Preferably, the solution is a borate buffered or phosphate buffered saline solution or deionized water. The particularly preferred solution contains about 500 ppm to about 18,500 ppm sodium borate, most particularly preferred about 1000 ppm of sodium borate.

If the anti-allergic agents are subject to oxidative degradation, agents that stabilize solutions containing such anti-allergic agents may be added. Such “oxidative stabilization agents” include but are not limited to chelants such as EDTA, Dequest, Desferal, silica, chitin derivatives such as chitosan, cellulose and its derivatives, and N,N,N′,N′,N″, N″-hexa(2-pyridyl)-1,3,5-tris(aminomethyl)benzene, and certain macrocyclic ligands such as crown ethers, ligand containing knots and catenands. See, David A. Leigh et al Angew. Chem Int. Ed., 2001, 40, No. 8, pgs. 1538-1542 and Jean-Claude Chambron et al. Pure & Appl. Chem., 1990, Vol. 62, No. 6, pgs. 1027-1034. Oxidative stabilization agents may include other compounds that inhibit oxidations such as those selected from the group consisting of 2,2′,2″,6,6′,6″-Hexa-(1,1-dimethylethyl)4,4′,4″-[(2,4,6-trimethyl-1,3,5-benzenetriyl)-trismethylene]-triphenol (Irganox 1330), 1,3,5tris[3,5-di(1,1-dimethylethyl)4-hydroxybenzyl]-1H,3H,5H-1,3,5-triazine-2,4,6-trione, pentaerythrityl tetrakis[3-[3,5-di(1,1-dimethylethyl)-4-hydroxyphenyl]-propionate], octadecyl-3-[3,5-di(1,1-dimethylethyl)-4-hydroxyphenyl]-propionate, tris[2,4-di(1,1-dimethylethyl)-phenyl]-phosphite, 2,2′-di(octadecyloxy)-5,5′-spirobi(1,3,2-dioxaphosphorinane), dioctadecyl disulphide, didodecyl-3,3′-thiodipropionate, dioctadecyl-3,3′-thiodipropionate, butylhydroxytoluene, ethylene bis[3,3-di[3-(1,1-dimethylethyl)-4-hydroxyphenyl]butyrate] and mixtures thereof. The preferred oxidative stabilization agents are diethylenetriaminepentaacetic acid (“DTPA”), or salts of DTPA such as CaNa₃DTPA, ZnNa₃DTPA, and Ca₂DTPA. See, U.S. App. Pat. No. 60/783,557 filed on, Mar. 17, 2006, entitled “Methods for Stabilizing Oxidatively Unstable Pharmaceutical Compositions” and its corresponding non-provisional filing which are hereby incorporated by reference in their entirety. If oxidative stabilization agents are added, it is preferred that at the concentration of oxidative stabilization agents in the solution be from about 2.5 pmoles/liter to about, 5000 pmoles/liter more preferably from about 20 pmoles/liter to about 1000 pmoles/liter, more preferably from about 100 pmoles/liter to about 1000 pmoles/liter, most preferably from about 100 pmoles/liter to about 500 pmoles/liter.

Other components may be added to the solutions which include but are not limited to antioxidants (radical scavengers), demulcents, antibacterial agents, solubilizers, surfactants, buffer agents, tonicity adjusting agents, chelating agents, preservatives, wetting agents, thickeners, water, saline solution, mineral oil, petroleum jelly, water soluble solvents, such as C₁₅₋₂₀ alcohols, C₁₅₋₂₀ amides, C₁₅₋₂₀ alcohols substituted with zwitterions, vegetable oils or mineral oils comprising from 0.5 to 5% by weight hydroxyethylcellulose, ethyl oleate, carboxymethylcellulose, polyvinyl-pyrrolidone and other non-toxic water-soluble polymers for ophthalmic uses, such as, for example cellulose derivatives, such as methylcellulose, alkali metal salts of carboxy-methylcellulose, hydroxymethylcellulose, hydroxyethylcellulose, methylhydroxypropyl-cellulose, hydroxypropylcellulose, chitosan and scleroglucan, acrylates or methacrylates, such as salts of poly(acrylic acid) or ethyl acrylate, polyacrylamides, natural products, such as gelatin, alginates, pectins, tragacanth, karaya gum, xanthan gum, carrageenin, agar and acacia, starch derivatives, such as starch acetate and hydroxypropyl starch, and also other synthetic products, such as poloxamers, e.g. Poloxamer F127, polyvinyl alcohol, polyvinylpyrrolidone, polyvinyl methyl ether, polyethylene oxide, preferably cross-linked poly(acrylic acid), such as neutral Carbopol, or mixtures of those polymers.

As used herein, “ophthalmic device” refers to an object that resides in or on the eye. These devices can provide optical correction or may be cosmetic. Ophthalmic devices include but are not limited to soft contact lenses, intraocular lenses, overlay lenses, ocular inserts, punctual plugs, and optical inserts. The preferred ophthalmic devices of the invention are soft contact lenses which are used to correct refractive errors, such as myopia, hyperopia, astigmatism and presbyopia or which are used for cosmetic purposes such as tinted lenses or other eye conditions such as keratoconus. The more preferred ophthalmic devices of the invention are soft contact lenses made from silicone elastomers or hydrogels, which include but are not limited to silicone hydrogels, and fluorohydrogels and excludes ophthalmic devices that contain phosphate group-containing methacrylates (i.e. CH₂—C(CH₃)—C(O)—(CH₂)ₙ—O—P(O)(OH)₂, where n is 1-4; CH₂C—C(CH₃)—C(O)—(C H₂)₂—O—P(O)(OH)—O—(CH₂)₂—O—C(O)—C(CH₃)—CH₂) or pre-polymers as such defined by US Pat. Application Publication No. US 2006/0100408. Soft contact lens formulations are disclosed in U.S. Pat. No. 5,710,302, WO 9421698, EP 406161, JP 2000016905, U.S. Pat. No. 5,998,498, U.S. Pat. No. 6,087,415, U.S. Pat. No. 5,760,100, U.S. Pat. No. 5,776, 999, U.S. Pat. No. 5,789,461, U.S. Pat. No. 5,849,811, and U.S. Pat. No. 5,965,631. The foregoing references are hereby incorporated by reference in their entirety. The particularly preferred ophthalmic devices of the inventions are prepared from formulations known by the United States Approved Names of acofilcon A, alofilcon A, alphafilcon A, amifilcon A, astifilcon A, atalafilcon A, balafilcon A, bisfilcon A, bufilcon A, comfilcon, crofilcon A, cyclofilcon A, darfilcon A, deltafilcon A, deltafilcon B, dimefilcon A, drooxifilcon A, epsifilcon A, esterifilcon A, etafilcon A, focofilcon A, galyfilcon A, genfilcon A, govafilcon A, hefilcon A, hefilcon B, hefilcon D, hilafilcon A, hilafilcon B, hioxifilcon B, hioxifilcon C, hixoifilcon A, hydrofilcon A, lenefilcon A, licryfilcon A, licryfilcon B, lidofilcon A, lidofilcon B, lotrafilcon A, lotrafilcon B, mafilcon A, mesifilcon A, methafilcon B, mipafilcon A, nelfilcon A, netrafilcon A, ocufilcon A, ocufilcon B, ocufilcon C, ocufilcon D, ocufilcon E, ofilcon A, omafilcon A, oxyfilcon A, pentafilcon A, perfilcon A, pevafilcon A, phemfilcon A, polymacon, senofilcon A, silafilcon A, siloxyfilcon A, tefilcon A, tetrafilcon A, trifilcon A, vasurfilcon, vifilcon, and xylofilcon A. More particularly preferred ophthalmic devices of the invention are made from the following formulations genfilcon A, lenefilcon A, comfilcon, lotrafilcon A, lotraifilcon B, and balafilcon A. More preferred lenses are made from the following formulations comfilcon, etafilcon A, galyfilcon A, senofilcon A, nelfilcon A, hilafilcon, tetrafilcon A, vasurfilcon, vifilcon, and polymacon. The most preferred lenses include those made from the etafilcon A formulation.

The preferred ophthalmic devices are devices to which one can add a minimum effective amount of an anti-allergic agent to a polymerized ophthalmic device, more preferably to a polymerized ophthalmic device that is hydrated with an aqueous solution to reach its equilibrium concentration. Polymerization refers to the process in which components of an ophthalmic device including but not limited to monomers, pre-polymers, diluents, catalysts, initiators, tints, UV blockers, antibacterial agents, polymerization inhibitors, and the like are reacted by thermal, chemical, and light initiated curing techniques to produce a formed polymer. The preferred methods of polymerization are the light initiated techniques disclosed in U.S. Pat. No. 6,822,016 which is hereby incorporated by reference in its entirety.

The particularly preferred ophthalmic devices contained a minimum effective amount of an anti-allergic agent prior to use by a patient and now due to such use, the ophthalmic device contains less than an minimum effective amount of the anti-allergic agent. The amount that is less than the minimum effective amount varies depending upon the anti-allergic agent. For example if the anti-allergic agent is ketotifen, the amount of ketotifen is less than the minimum effective amount, namely less than about 9 μg of ketotifen. Preferably the amount of ketotifen fumarate is less than about 9 μg of ketotifen and more than about 18 nanograms of ketotifen.

Further the invention includes a method of preparing an ophthalmic device comprising an minimum effective amount of an anti-allergic agent comprising instructing a patient or an ophthalmic professional to treat an ophthalmic device comprising less than about the minimum effective amount of an anti-allergic agent with a solution comprising said anti-allergic agent, wherein the amount of said anti-allergic agent in said solution exceeds the minimum effective amount. The terms ophthalmic device minimum effective amount, anti-allergic agent, solution, treat, and exceeds the minimum effective amount have their aforementioned meanings and preferred ranges. The term “ophthalmic professional” includes opticians, ophthalmologists, optometrists, and manufacturers of ophthalmic devices.

Still further the invention includes a kit comprising an ophthalmic device comprising a minimum effective amount of an anti-allergic agent and a solution comprising said anti-allergic agent, wherein the amount of said anti-allergic agent in said solution exceeds the minimum effective amount. The terms ophthalmic device minimum effective amount, anti-allergic agent, solution, and exceeds the minimum effective amount have their aforementioned meanings and preferred ranges. The term “kit” includes a single unit package that contains at least one ophthalmic device and a container of a solution comprising said anti-allergic agent.

Yet still further, the invention includes a method of preparing an ophthalmic device comprising an minimum effective amount of an anti-allergic agent comprising treating an ophthalmic device that does not comprise an anti-allergic agent with a solution comprising said anti-allergic agent for at least about 15 minutes, wherein the amount of said anti-allergic agent in said solution exceeds the minimum effect amount. The terms ophthalmic device minimum effective amount, anti-allergic agent, exceeds the minimum effective amount, treating and solution all have their aforementioned meanings and preferred ranges. It is preferred that the ophthalmic device that does not comprise an anti-allergic agent is treated with the solution for greater than about two hours to about 24 hours, more preferably greater than about two hours to about 16 hours, most preferably greater than about two hours to about 12 hours.

Still yet further, the invention includes a method of preparing an ophthalmic device comprising an minimum effective amount of an anti-allergic agent comprising instructing a patient or an ophthalmic professional to treat an ophthalmic device that does not comprise an anti-allergic agent with a solution comprising said anti-allergic agent for at least about 15 minutes, wherein the amount of said anti-allergic agent exceeds the minimum effective amount. The terms ophthalmic device minimum effective amount, anti-allergic agent, exceeds the minimum effective amount, treat, and solution all have their aforementioned meanings and preferred ranges.

Yet further still the invention includes a kit comprising an ophthalmic device that does not comprise an anti-allergic agent and a solution comprising said anti-allergic agent, wherein the amount of said anti-allergic agent in said solution exceeds the minimum effective amount. The terms ophthalmic device minimum effective amount, anti-allergic agent, exceeds the minimum effective amount, and solution all have their aforementioned meanings and preferred ranges.

The advantages of the invention may be found in reasons related to manufacturing cost, storing too many lenses in the doctor's office, and requiring a patient with seasonal allergies to maintain a stock of ophthalmic lenses with and without antihistamine. For example, ophthalmic lenses containing anti-allergic agents may not be available in all optical prescriptions ex. torics, bifocals, or wearing modalities and schedules ex. Daily wear, extended wear, frequent replacement. Therefore, it would be beneficial to provide a method and solution so that the anti-allergic agent may be used with the user's current lenses. A method and solution that eliminate the need for duplicate lenses would be economically beneficial.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

### Example 1

### Preparation of Ophthalmic Devices Ketotifen Fumarate

Six lots of 1-Day Acuvue® Brand Contact Lenses (etafilcon A) were cured and hydrated with deionized water. These lenses were inserted into blister packages containing 950 uL of a 43 ug/mL ketotifen fumarate packing solution per lens (packing solution formulation: borate buffered saline containing 0.83% sodium chloride, 0.9% boric acid, 100 ug/mL of dicalcium DTPA, and 0.1% sodium borate decahydrate ). The blister packaged were sealed with an aluminum foil laminate lidstock. They were sterilized once (1×) (124C, 18 minute exposure )and assayed (as described below) for the lens ketotifen content. The number of micrograms per lens are listed in column A of Table 1

### Extraction/Assay Procedure

Packages of lenses are opened, blotted and transferred to a scintillation vial using tweezers. Three mL of Eluent A (defined below) are added and the vials were sonicated for 1 hour at ambient conditions. The lenses were removed from the scintillation vials and the remaining solution was analyzed for ketotifen content by HPLC.

Two eluent solutions, and a stock ketotifen fumarate solution were used in the assay, having the following compositions Eluent A-17% acetonitrile in 0.025 postassium phosphate, monobasic buffer 0.2% triethylamine, 0.13% o-phosphoric acid (balance deionized water): Eluent B-50% acetonitrile in 0.025 postassium phosphate, monobasic buffer 0.2% triethylamine, 0.13% o-phosphoric acid (balance deionized water), and ketotifen fumarate stock standard 72.72% ketotifen fumarate (balance Eluent A).

The HPLC uses an Agilent Zorbax Exlipse WDB-18 Rapid Resolution HT 4.6 mm×1.8μ Guard Column: Phenomenex HPLC Guard Cartridge System “Security Guard” and the dector has a wavelength of 299 nm, a VW detector peak width Setting: “>0.05 min”, a Flow rate of 1.0 mL/min, and an injection volume of 100 μL. The number of micrograms of ketotifen per lens were analyzed by comparing the peak area of the extracted solutions versus peak area of against peak areas of the ketotifen fumarate stock standard and using standard equations.

Six lenses were removed from their respective containers, blotted, and transferred to scintillation vials. Five mL of Tear Solution (defined below) was added and the vials were shaken for five hours at room temperature. The extraction process was repeated two more times by replacing the tear solution with a fresh 5 mL volume and additional shaking for five hours each time. The lenses were extracted and analyzed for ketotifen content by the extraction/assay procedure above. The amount of ketotifen in those lenses is presented in column B of Table 1

Three of the lenses were extracted with tear solution and placed in blister packages with 950 uL of a 43 ug/mL ketotifen fumarte packing solution as described in Example 1, sealed, and allowed to equilibrate for 12 hrs at room temperature. After this equilibration, the lenses were analyzed for ketotifen following the extraction/assay procedure above. The amount of ketotifen in those lenses is listed in column C of Table 1. This experiment shows lenses that lose their ketotifen absorb ketotifen after treatment following the methods of this invention.

TABLE 1

| Lens | | | |
|---|---|---|---|
| Lot # | A | B | C |
| 1 | 18.348 | 0.375 | 18.399 |
| 2 | 18.408 | 0.342 | 19.353 |
| 3 | 17.901 | 0.333 | 18.990 |
| 4 | 18.378 | | |
| 5 | 18.063 | | |
| 6 | 17.841 | | |
| Average | 18.157 | 0.350 | 18.914 |
| Std dev | 0.254 | 0.022 | 0.482 |

### Example 2

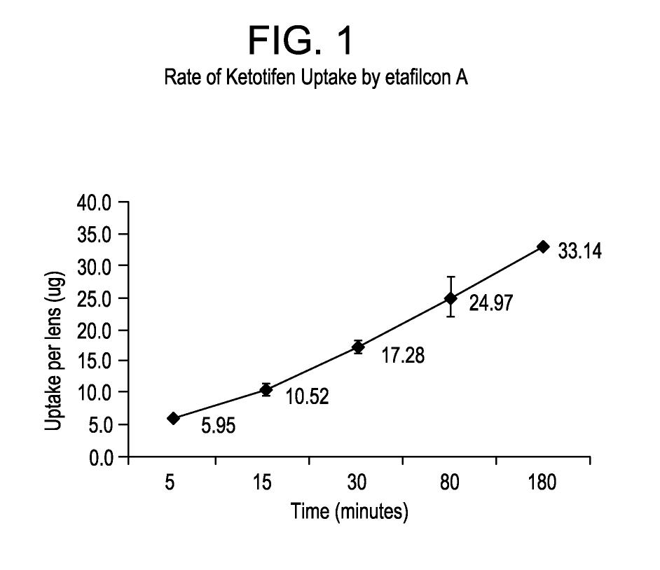

Three lots of 1-Day Acuvue® Brand Contact Lenses (etafilcon A) were cured and hydrated with deionized water. These lenses were inserted into glass vials containing 3.0 mL of a 43 ug/mL ketotifen fumarate packing solution per lens (packing solution formulation: borate buffered saline containing 0.83% sodium chloride, 0.9% boric acid, 100 ug/mL of dicalcium DTPA, and 0.1% sodium borate decahydrate ). The soaked and harvested at 5, 15, 30, 90 and 180 minutes and extracted and assayed as in Example 1 to determine the amount of ketotifen per treated lens. The results were tabulated and presented in FIG. 1. This graph illustrates that a minimum effective amount of in an etafilcon A contact lens is attained within 15 minutes of treatment.

What is claimed is

1. A method of preparing an ophthalmic device comprising an minimum effective amount of an anti-allergic agent comprising treating an ophthalmic device comprising less than about a minimum effective amount of an anti-allergic agent with a solution comprising said anti-allergic agent, wherein the amount of said anti-allergic agent in said solution exceeds the minimum effective amount

2. The method of claim 1 wherein the anti-allergic agent is selected from the group consisting of azelastine, epinastine, ketotifen, ketotifen fumarate, norketotifen fumarate, olopatadine and mixtures thereof.

3. The method of claim 1 wherein the anti-allergic agent is selected from the group consisting of ketotifen and its pharmaceutically acceptable salts.

4. The method of claim 1 wherein the anti-allergic agent is ketotifen or its pharmaceutically acceptable salts and the minimum effective amount of ketotifen or its pharmaceutically acceptable salts is about 9 μg to about 40 μg.

5. The method of claim 1 wherein the anti-allergic agent is ketotifen or its pharmaceutically acceptable salts and the minimum effective amount of ketotifen or its pharmaceutically acceptable salts is about 9 μg to about 20 μg.

6. The method of claim 1 wherein the minimum effective amount is exceeded by between about 0.1% and about 50%, in a volume of solution that is between about 500 μL and about 3000 μL

7. The method of claim 1 wherein the ophthalmic device is a soft contact lens.

8. The method of claim 1 wherein the anti-allergic agent is ketotifen or its pharmaceutically acceptable salts, the minimum effective amount of ketotifen or its pharmaceutically acceptable salts is about 9 μg to about 40 μg and the ophthalmic device is a soft contact lens comprising etafilcon A.

9 The method of claims 8 wherein the amount of anti-allergic agent in the solution exceeds the minimum effective amount by about 50% in about 1000 μL of the solution.

10. The method of claim 1 wherein the ophthalmic device is treated for at least 15 minutes.

11. A kit comprising an ophthalmic device comprising a minimum effective amount of an anti-allergic agent and a solution comprising said anti-allergic agent, wherein the amount of said anti-allergic agent in said solution exceeds the minimum effective amount.

12. The kit of claim 11 wherein the anti-allergic agent is selected from the group consisting of azelastine, epinastine, ketotifen, ketotifen fumarate, norketotifen fumarate, olopatadine and mixtures thereof.

13. The kit of claim 11 wherein the anti-allergic agent is selected from the group consisting of ketotifen and its pharmaceutically acceptable salts.

14. The kit of claim 11 wherein the anti-allergic agent is ketotifen or its pharmaceutically acceptable salts and the minimum effective amount of ketotifen or its pharmaceutically acceptable salts is about 9 μg to about 40 μg.

15. The kit of claim 11 wherein the anti-allergic agent is ketotifen or its pharmaceutically acceptable salts and the minimum effective amount of ketotifen or its pharmaceutically acceptable salts is about 9 μg to about 20 μg.

16. The kit of claim 11 wherein the minimum effective amount is exceeded by between about 0.1% and about 50%, in a volume of solution that is between about 500 μL and about 3000 μL

17. The kit of claim 11 wherein the ophthalmic device is a soft contact lens.

18. The kit of claim 11 wherein the anti-allergic agent is ketotifen or its pharmaceutically acceptable salts, the minimum effective amount of ketotifen or its pharmaceutically acceptable salts is about 9 μg to about 40 μg and the ophthalmic device is a soft contact lens comprising etafilcon A.

19. The kit of claims 18 wherein the amount of anti-allergic agent in the solution exceeds the minimum effective amount by about 50% in about 1000 μL of the solution.

20. A method of preparing an ophthalmic device comprising an minimum effective amount of an anti-allergic agent comprising instructing a patient or an ophthalmic professional to treat an ophthalmic device comprising less than about the minimum effective amount of an anti-allergic agent with a solution comprising said anti-allergic agent, wherein the amount of said anti-allergic agent in said solution exceeds the minimum effective amount.

21. The method of claim 20 wherein the anti-allergic agent is selected from the group consisting of azelastine, epinastine, ketotifen, ketotifen fumarate, nor-ketotifen fumarate, olopatadine and mixtures thereof.

22. The method of claim 20 wherein the anti-allergic agent is selected from the group consisting of ketotifen and its pharmaceutically acceptable salts.

23. The method of claim 20 wherein the anti-allergic agent is ketotifen or its pharmaceutically acceptable salts and the minimum effective amount of ketotifen or its pharmaceutically acceptable salts is about 9 μg to about 40 μg.

24. The method of claim 20 wherein the anti-allergic agent is ketotifen or its pharmaceutically acceptable salts and the minimum effective amount of ketotifen or its pharmaceutically acceptable salts is about 9 μg to about 20 μg.

25. The method of claim 20 wherein the minimum effective amount is exceeded by between about 0.1% and about 50%, in a volume of solution that is between about 500 μL and about 3000 μL

26. The method of claim 20 wherein the ophthalmic device is a soft contact lens.

27. The method of claim 20 wherein the anti-allergic agent is ketotifen or its pharmaceutically acceptable salts, the minimum effective amount of ketotifen or its pharmaceutically acceptable salts is about 9 μg to about 40 μg and the ophthalmic device is a soft contact lens comprising etafilcon A.

28. The method of claim 20 wherein the amount of anti-allergic agent in the solution exceeds the minimum effective amount by about 50% in about 1000 μL of the solution.

29. The method of claim 20 wherein the ophthalmic device is treated for at least 15 minutes.

30. A method of preparing an ophthalmic device comprising an minimum effective amount of an anti-allergic agent comprising treating an ophthalmic device that does not comprise an anti-allergic agent with a solution comprising said anti-allergic agent for at least about 15 minutes, wherein the amount of said anti-allergic agent in said solution exceeds the minimum effect amount.

31. The method of claim 30 wherein the anti-allergic agent is selected from the group consisting of azelastine, epinastine, ketotifen, ketotifen fumarate, norketotifen fumarate, olopatadine and mixtures thereof.

32. The method of claim 30 wherein the anti-allergic agent is selected from the group consisting of ketotifen and its pharmaceutically acceptable salts.

33. The method of claim 30 wherein the anti-allergic agent is ketotifen or its pharmaceutically acceptable salts and the minimum effective amount of ketotifen or its pharmaceutically acceptable salts is about 9 μg to about 40 μg.

33. The method of claim 30 wherein the anti-allergic agent is ketotifen or its pharmaceutically acceptable salts and the minimum effective amount of ketotifen or its pharmaceutically acceptable salts is about 9 μg to about 20 μg.

34. The method of claim 30 wherein the minimum effective amount is exceeded by between about 0.1% and about 50%, in a volume of solution that is between about 500 μL and about 3000 μL

35. The method of claim 30 wherein the ophthalmic device is a soft contact lens.

36. The method of claim 30 wherein the anti-allergic agent is ketotifen or its pharmaceutically acceptable salts, the minimum effective amount of ketotifen or its pharmaceutically acceptable salts is about 9 μg to about 40 μg and the ophthalmic device is a soft contact lens comprising etafilcon A.

37. The method of claims 36 wherein the amount of anti-allergic agent in the solution exceeds the minimum effective amount by about 50% in about 1000 μL of the solution.

38. The method of claim 30 wherein the ophthalmic device is treated for about 2 to about 16 hours.

39. A method of preparing an ophthalmic device comprising an minimum effective amount of an anti-allergic agent comprising instructing a patient or an ophthalmic professional to treat an ophthalmic device that does not comprise an anti-allergic agent with a solution comprising said anti-allergic agent for at least about 15 minutes, wherein the amount of said anti-allergic agent exceeds the minimum effective amount.

40. The method of claim 39 wherein the anti-allergic agent is selected from the group consisting of azelastine, epinastine, ketotifen, ketotifen fumarate, norketotifen fumarate, olopatadine and mixtures thereof.

41. The method of claim 39 wherein the anti-allergic agent is selected from the group consisting of ketotifen and its pharmaceutically acceptable salts.

42. The method of claim 39 wherein the anti-allergic agent is ketotifen or its pharmaceutically acceptable salts and the minimum effective amount of ketotifen or its pharmaceutically acceptable salts is about 9 μg to about 40 μg.

43. The method of claim 39 wherein the anti-allergic agent is ketotifen or its pharmaceutically acceptable salts and the minimum effective amount of ketotifen or its pharmaceutically acceptable salts is about 9 μg to about 20 μg.

44. The method of claim 39 wherein the minimum effective amount is exceeded by between about 0.1% and about 50%, in a volume of solution that is between about 500 μL and about 3000 μL

45. The method of claim 39 wherein the ophthalmic device is a soft contact lens.

46. The method of claim 39 wherein the anti-allergic agent is ketotifen or its pharmaceutically acceptable salts, the minimum effective amount of ketotifen or its pharmaceutically acceptable salts is about 9 μg to about 40 μg and the ophthalmic device is a soft contact lens comprising etafilcon A.

47. The method of claims 46 wherein the amount of anti-allergic agent in the solution exceeds the minimum effective amount by about 50% in about 1000 μL of the solution.

48. The method of claim 39 wherein the ophthalmic device is treated for about 2 to about 16 hours.

49. A kit comprising an ophthalmic device that does not comprise an anti-allergic agent and a solution comprising said anti-allergic agent, wherein the amount of said anti-allergic agent in said solution exceeds the minimum effective amount.

50. The method of claim 49 wherein the anti-allergic agent is selected from the group consisting of azelastine, epinastine, ketotifen, ketotifen fumarate, nor-ketotifen fumarate, olopatadine and mixtures thereof.

51. The method of claim 49 wherein the anti-allergic agent is selected from the group consisting of ketotifen and its pharmaceutically acceptable salts.

52. The method of claim 49 wherein the anti-allergic agent is ketotifen or its pharmaceutically acceptable salts and the minimum effective amount of ketotifen or its pharmaceutically acceptable salts is about 9 μg to about 40 μg.

53. The method of claim 49 wherein the anti-allergic agent is ketotifen or its pharmaceutically acceptable salts and the minimum effective amount of ketotifen or its pharmaceutically acceptable salts is about 9 μg to about 20 μg.

54. The method of claim 49 wherein the minimum effective amount is exceeded by between about 0.1% and about 50%, in a volume of solution that is between about 500 μL and about 3000 μL

55. The method of claim 49 wherein the ophthalmic device is a soft contact lens.

56. The method of claim 49 wherein the anti-allergic agent is ketotifen or its pharmaceutically acceptable salts, the minimum effective amount of ketotifen or its pharmaceutically acceptable salts is about 9 μg to about 40 μg and the ophthalmic device is a soft contact lens comprising etafilcon A.

57. The method of claims 49 wherein the amount of anti-allergic agent in the solution exceeds the minimum effective amount by about 50% in about 1000 μL of the solution.
